(19) United States
(12) Patent Application Publication
(10) Pub. No.: US 2024/0341869 A1
(43) Pub. Date: Oct. 17, 2024

(54) SURGICAL ROBOTIC MANIPULATOR PROVIDING ENHANCED DEXTERITY ACROSS THE SURGICAL WORKSPACE

(71) Applicants: Federico Campisano, Timnath, CO (US); Kevin Andrew Hufford, Durham, NC (US); Paul Wilhelm Schnur, Pittsboro, NC (US)

(72) Inventors: Federico Campisano, Timnath, CO (US); Kevin Andrew Hufford, Durham, NC (US); Paul Wilhelm Schnur, Pittsboro, NC (US)

(21) Appl. No.: 18/582,601

(22) Filed: Feb. 20, 2024

Related U.S. Application Data
(60) Provisional application No. 63/485,906, filed on Feb. 19, 2023.

(51) Int. Cl. A61B 34/30; A61B 17/29

(52) U.S. Cl. CPC A61B 34/30; A61B 17/29

(57) ABSTRACT

A surgical robotic manipulator includes a proximal assembly having a vertical column and a horizontal boom, and proximal joints in a prismatic-revolute-prismatic configuration allowing the horizontal boom to be raised, lowered and rotated about the vertical axis of the column. A distal assembly carries an instrument receiver configured to receive a surgical instrument. Joints of the distal assembly are configured to maneuver the surgical instrument in an additional four degrees of freedom.

### RELATED APPLICATIONS

This application claims the benefit of U.S. Provisional Application No. 63/485,906, filed Feb. 19, 2023.

### TECHNICAL FIELD OF THE INVENTION

The present invention relates generally to the field of surgical devices and systems, and more particularly to the field of robotic manipulators for robot-assisted surgery.

### BACKGROUND

There are various types of surgical robotic systems on the market or under development. Some surgical robotic systems use a plurality of robotic manipulators or arms. Each manipulator carries a surgical instrument, or the camera used to capture images from within the body for display on a monitor. Typical configurations allow two or three instruments and the camera to be supported and manipulated by the system. Input to the system is generated based on input from a surgeon positioned at a surgeon console, typically using input devices such as input handles. The system responds to movement of a user input device by controlling the robotic manipulator that is associated with that input device to position, orient and actuate the surgical instrument positioned on that manipulator. The image captured by the camera is shown on a display at the surgeon console. The console may be located patient-side, within the sterile field, or outside of the sterile field.

Each robotic manipulator includes a portion, typically at the terminal end of the arm, that is designed to support and operate a surgical device assembly. The surgical device assembly includes a surgical instrument having a shaft and a distal end effector on the shaft. The end effector is positionable within a patient. The end effector may be one of many different types that are used in surgery including, without limitation, end effectors having one or more of the following features: jaws that open and close, a section at the distal end of the shaft that bends or articulates in one or more degrees of freedom, a tip that rolls axially relative to the shaft, a shaft that rolls axially relative to the manipulator arm.

In many robotic surgical systems, including those using rigid shaft instruments, the surgical instruments are both robotically manipulated by the robotic manipulator arms disposed outside the patient's body, as well as electromechanically actuated within the patient's body. In many surgical systems, robotic manipulation using the arm pivots the instrument shaft relative to the incision site on the patient, and may also alter the insertion depth of the instrument into the patient and/or cause the instrument to roll about its longitudinal axis. Additionally, electromechanical actuation (or hydraulic/pneumatic actuation) may open and close jaws of the instrument, and/or actuate articulating or bending of the distal end of the instrument shaft, and/or roll the instrument's shaft or distal tip. Some systems may use only this latter form of instrument motion while holding the more proximal part of the instrument in a fixed position outside the body using a fixed support or inactive robotic manipulator.

A proximal housing is typically positioned on the proximal end of the instrument shaft. This housing functions as an adapter or interface between the surgical instrument and the robotic manipulator. The adapter may include passive actuation mechanisms that receive motion transferred from the active actuators in the robotic manipulator or other instrument support to drive functions of the instrument end effector. Such functions may include jaw open-close, shaft articulating or bending, or other functions. As noted above, the instrument actuators for driving the motion of the end effector, which respond to user input to cause actuation of the instrument's functions, are normally electromechanical motors or other types of motors, but could also be hydraulic or pneumatic. They are often positioned in the terminal portion of the robotic manipulator. In some cases, they are positioned in the proximal housing of the surgical device assembly. In still other configurations, some are in the proximal housing while others are in the robotic manipulator. In the latter example, some functions of the end effector might be driven using one or more motors in the terminal portion of the manipulator while other motion might be driven using motors in the proximal housing. See, for example, US 2016/0058513, Surgical System with Sterile Wrappings, in which jaw open-close functions are initiated using electromechanical actuators in the robotic manipulator, and in which rotation or swivel functions of the instrument are initiated using electromechanical actuators housed in the proximal housing of the surgical device assembly.

Various systems have different types of mechanical interfaces between the adapter and the robotic manipulator. It is through these interfaces that motion generated by the instrument actuators within the robotic manipulator is communicated to one or more mechanical inputs of the adapter to control degrees of freedom of the instrument and, if applicable, its jaw open-close function. This motion may be communicated through a sterile drape positioned between the sterile adapter and the non-sterile manipulator arm. In some commercially available robotic systems, the motion is communication using rotary connections in which rotating disks on the manipulator transfer motion to rotating disks on an instrument adapter. Sec, for example, the configuration shown in U.S. Pat. No. 6,491,701. In others, such as the embodiment shown in U.S. Pat. No. 9,358,682, a transverse slider pin extends laterally from one side of the case mounted to the proximal end of the instrument. It is moveable to open and close jaws of the instrument (FIG. 18 of the patent). When the instrument is mounted to the manipulator arm, the slider pin is received by a corresponding component 430 (FIG. 19) in the manipulator arm. When it is necessary to open/close the instrument jaws, the component 430 is translated on a carriage by motors in the laparoscopic instrument actuator 400 of the manipulator arm. This advances the slider pin 314 to actuate the jaws.

Yet another adapter configuration is shown and described in commonly owned US Publication No. 2021/169595. In that configuration, the adapter of a surgical instrument includes a pair of planar faces on opposite sides of the adapter. Two longitudinally slidable drive inputs are exposed at each face, giving the adapter a total of four drive inputs. The instrument is engaged to a robotic manipulator by positioning the instrument adapter between two arms of an expandable instrument drive system (“IDS”), and then closing the arms to capture the adapter between the arms. This engages each drive input with a corresponding drive output on the instrument drive system. The use of four drive inputs in this configuration allows for both jaw open-close functions and articulation of the end effector in both pitch and yaw directions.

The instruments are exchangeable during the course of the procedure, allowing one instrument (with its corresponding adapter) to be removed from a manipulator and replaced with another instrument and its corresponding adapter.

In some systems, some of the surgical instruments may be removably connected to their respective adapters. This facilitates post-surgery cleaning and sterilization of the instruments and adapters, by allowing them to be separated for cleaning and sterile processing.

During robot-assisted minimally invasive surgeries (MIS), the robotic manipulators are required to perform highly precise and dexterous tasks. The surgical instruments are inserted into the patient body through a surgical device called a trocar, which is located at the incision site. For patient safety and to prevent injury at the incision site, a motion constraint is placed on lateral translation of the instrument shaft at the location of the trocar. The combination of both motion of the instrument distal-end and constraint at the trocar define the motion pattern for manipulators designed for MIS.

More specifically, each surgical instrument must pivot with respect to a remote center of motion (RCM) at the incision site. Accordingly, motion of the surgical instrument shaft is limited to pitch and yaw motion relative to the RCM, as well as translational motion along the instrument's longitudinal axis, and rotational or “roll” motion relative to the instrument's longitudinal axis.

Commercially available surgical robotic systems use different approaches to ensure that instrument motion is constrained relative to the RCM. One approach is to mechanically constrain instrument motion such that it occurs relative to the RCM. In other words, the mechanical structure of these manipulators constrains them to move the instrument with respect to a fulcrum. See, for example, the configuration shown in U.S. Pat. No. 6,491,701, which describes a parallelogram arrangement configured so that the instrument rotates around a point in space coinciding with the trocar. In such systems, the dexterity of the surgical instrument is not impacted by the constraint at the incision. Force and motion control of these systems need not consider motion relative to a remote center of motion (RCM) due to the mechanical restraints. However, the architecture required for these mechanical constraints can crowd the workspace surrounding the incision, resulting in a limited surgical workspace, which may leave limited room for surgical personnel, and increase potential for collisions between manipulators. Also, in setting up such a system, surgical personnel must ensure that the location of the RCM is aligned with the trocar, such as by physically coupling the trocar to the manipulator, a feature that requires use of trocars specially developed for use with the robotic system.

Other surgical robotic systems do not use mechanical constraints to restrict instrument motion to an RCM, but instead use algorithms to constrain such motion such that it occurs relative to the incision. U.S. Pat. No. 9,855,662, entitled Force Estimation for a Minimally Invasive Robotic Surgery System, describes a method by which input from a 6 DOF force/torque sensor on the robotic manipulator is used to determine the fulcrum/RCM about which the surgical instrument should be pivoted, which corresponds to the location of the incision along the instrument shaft. Motion of the robotic manipulator is thus algorithmically controlled to constrain the motion such that the instrument pivots relative to the RCM. Such systems lack the dimensional challenges of the mechanically constrained systems, can be easier to set up by the surgical team, have a simpler collision workspace, permit use of standard laparoscopic trocars, and allow the surgical team to more easily access the patient at the bedside. One such system is the Senhance System sold by Asensus Surgical, Inc.

Other systems using algorithmically constrained RCMs for robotic motion use alternative methods for determining the coordinates of the RCM at the incision site. For example, some systems position the tip of a surgical instrument or other probe at the trocar site. The surgical instrument or probe is mounted to the robotic arm. The system is instructed to calculate the coordinates of the RCM using, for example, the known length of the instrument or probe, and the joint positions of the robotic arm during the RCM-setting step.

As discussed above, the desired motion of the distal-end of the surgical instrument is commanded by the surgeon through the user inputs. The robotic manipulator on the patient side replicates the motion commanded by the surgeon on the instrument. The quality of this replicated motion (i.e. the ability to produce the desired instrument end-effector velocity) greatly depends on the kinematic representation of the manipulator, location of the constraint, the relative orientation of the patient, and the dimension of the internal surgical workspace (i.e. surgical reach) required to accomplish the procedure.

This application describes an algorithmically constrained robotic manipulator that can provide dexterity on the level achieved using mechanically constrained manipulators, while allowing the case of set up, simple collision workspace, and the bedside patient access afforded by prior art algorithmically constrained manipulators.

This application describes a redundant robotic manipulator having a kinematic structure that allows user input motion to be accurately replicated motion by maintaining high dexterity and sensitivity across the overall surgical workspace. It further describes control methods that apply to the aforementioned kinematic framework for enhancing dexterity and sensitivity across the manipulator workspace independent of the preoperative configuration of the arm and the patient.

### BRIEF DESCRIPTION OF THE DRAWINGS

FIG. 1 is a perspective view of a robot-assisted surgical system comprising four robotic manipulator arms of the type described in this application.

FIG. 2 is a perspective view of a robotic manipulator arm with the receiver/IDS and instrument assembly mounted to it.

FIG. 3 is a perspective view showing the IDS of FIG. 2 and the surgical instrument separated from the IDS.

FIG. 4 is a schematic representation of the manipulator arm of FIG. 2, and illustrates the fulcrum relative to which motion of the surgical instrument is constrained.

FIGS. 5A and 5B are side elevation views showing the horizontal boom assembly of the manipulator arm of FIG. 2 at two different height positions resulting from isolated motion of joint J1. The instrument drive system and instrument are not shown, and the coverings containing the joints and other components are removed.

FIGS. 6A-6C are top plan views showing the horizontal boom assembly in three different rotational positions resulting from isolated motion of joint J2. The instrument drive system and instrument are not shown, and the coverings containing the joints and other components are removed.

FIGS. 7A-7B are side elevation views showing the horizontal boom assembly of the manipulator arm of FIG. 2 at two different translational positions relative to the vertical column, resulting from isolated motion of joint J3. The instrument drive system and instrument are not shown, and the coverings containing the joints and other components are removed.

FIG. 8A is a perspective view schematically illustrating a vertical plane containing the instrument shaft and a yaw axis; FIG. 8B is similar to FIG. 8A but shows the distal joints in a different orientation resulting from motion at J4.

FIG. 9 is a top perspective view illustrating the range of motion of joint J4. The instrument drive system and instrument are not shown, and the coverings containing the joints and other components are removed.

FIGS. 10A-10E are side elevation views showing examples of positions of the proximal link that can be from isolated motion of joint J5. The instrument drive system and instrument are not shown, and the coverings containing the joints and other components are removed.

FIGS. 11A-11D are side elevation views showing examples of positions of the distal link that can be from isolated motion of joint J6. The instrument drive system and instrument are not shown, and the coverings containing the joints and other components are removed.

FIGS. 12A and 12B schematically illustrate use of both link angle and approach angle as configuration variables for control of motion of the arm.

FIGS. 13A and 13B are similar to FIGS. 12A and 12B, but show that translation of the instrument along its longitudinal axis can be achieved.

FIG. 14 schematically represents motion control in which translational motion of J3 may be limited to prevent movement of the J4 axis beyond the RCM.

FIG. 15 shows an 8 degree of freedom alternative to the arm of FIG. 2.

FIG. 16 shows a second 8 degree of freedom alternative to the arm of FIG. 2.

### DETAILED DESCRIPTION

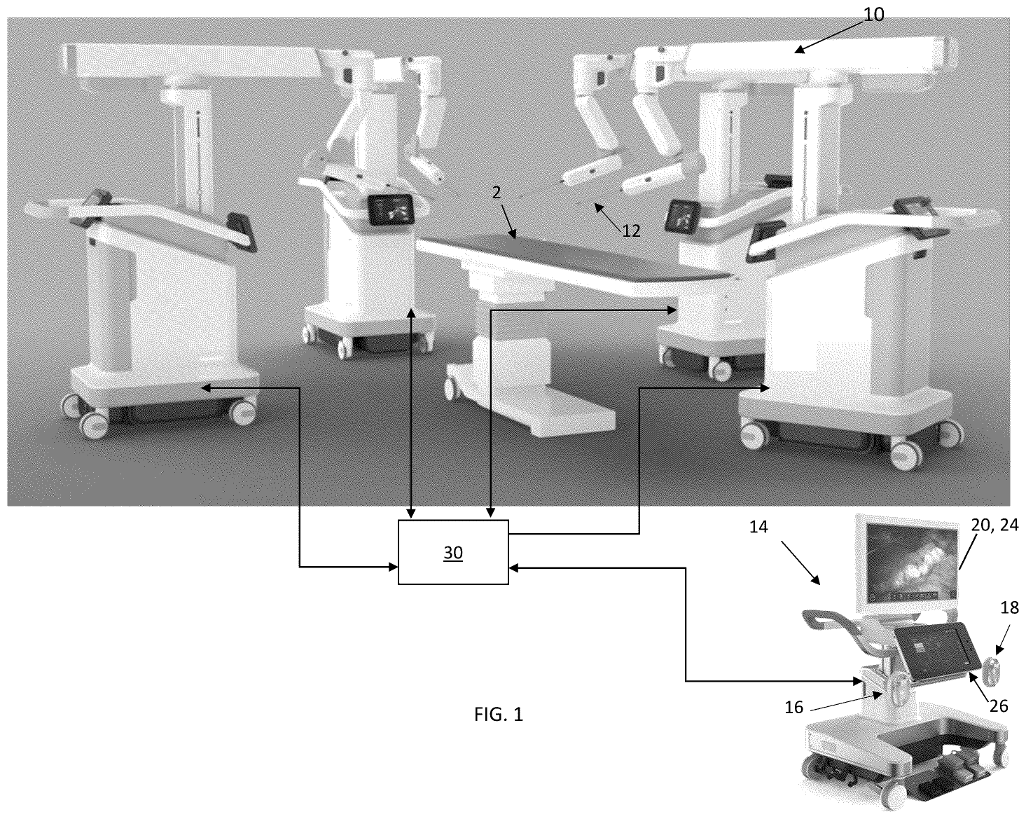

Although the concepts described herein may be used on a variety of robotic surgical systems, the embodiments will be described with reference to a system of the type shown in FIG. 1. In the illustrated system, robotic manipulators 10 are disposed adjacent to a patient bed 2. Each manipulator 10 is configured to maneuver a surgical instrument 12 which has a distal end effector positionable in a patient body cavity. FIG. 1 shows four robotic manipulators, although in other configurations, the number of manipulators may differ.

A surgeon console 14 has two input devices such as handles 16, 18. The input devices are configured to be manipulated by a user to generate signals that are used to command motion of the robotic manipulators in multiple degrees of freedom in order to maneuver the instrument end effectors within the body cavity. The input devices may be mounted to linkages, gimbals, etc. equipped with sensors that generate signals corresponding to positions or movement of the input devices in manners known to those skilled in the art. In other embodiments, the input devices may take the form of handles that are tracked using a tracking system, such as an optical tracking system or an electromagnetic tracking system, either alone or in combination with other sensors within the handles, such as IMUs etc.

In use, a user selectively assigns the two handles 16, 18 to two of the robotic manipulators 10, allowing surgeon control of two of the surgical instruments 12 at any given time. To control a third one of the instruments disposed at the working site, one of the two handles 16, 18 may be operatively disengaged from one of the initial two instruments and then operatively paired with the third instrument, or another form of input may control the third instrument as described in the next paragraph.

One of the instruments 12 is a camera that captures images of the operative field in the body cavity. The camera may be moved by its corresponding robotic manipulator using input from a variety of types of input devices, including, without limitation, one of the handles 16, 18, additional controls on the console, a foot pedal, an eye tracker 20, voice controller, etc. The console may also include a display or monitor 24 configured to display the images captured by the camera, and for optionally displaying system information, patient information, etc. An auxiliary display 26, which may be a touch screen display, can further facilitate interactions with the system.

The surgical system allows the operating room staff to remove and replace the surgical instrument 12 carried by a robotic manipulator 10, based on the surgical need. When an instrument exchange is necessary, surgical personnel remove an instrument from a manipulator arm and replace it with another.

As discussed, manipulation of the input devices 16, 18 results in signals that are processed by the system to generate instructions for commanding motion of the manipulators in order to move the instruments in multiple degrees of freedom and to, as appropriate, control operation of electromechanical actuators/motors that drive instrument functions such as articulation, bending, and/or actuation of the instrument end effectors. One or more control units 30 are operationally connected to the robotic arms and to the user interface. The control units receive user input that is generated as a result of movement of the input devices, and generates commands for the robotic arms to manipulate the surgical instruments so that the surgical instruments are positioned and oriented in accordance with the input provided by the user.

Sensors in the robotic manipulators determine the forces that are being applied to the patient by the robotic surgical tools during use. U.S. Pat. No. 9,855,662, entitled Force Estimation for a Minimally Invasive Robotic Surgery System, which is incorporated herein by reference, describes a method by which input from a 6 DOF force/torque sensor on the robotic manipulator is used to determine the RCM about which the surgical instrument should be pivoted, which corresponds to the location of the incision along the instrument shaft. Motion of the robotic manipulator is thus algorithmically controlled to constrain the motion such that the instrument pivots relative to the RCM. In the presently disclosed embodiments, a sensor of this type may be optionally positioned just proximal to the instrument drive system 104. It should be understood that use of the 6 DOF force/torque sensor to determine the RCM is desirable, but is not essential for practice of the present invention. Other methods for determining the location of the RCM, including those discussed in the Background, may be used in the presently-described manipulator arm in lieu of methods using the 6 DOF force/torque sensor.

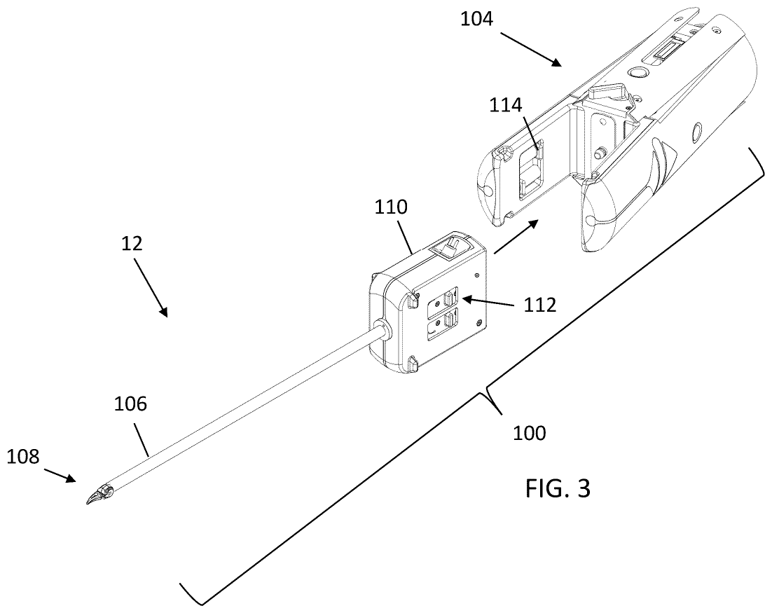

Referring to FIGS. 3, positioned at the distal end of each manipulator arm is a receiver 104, which may also be referred to as an instrument drive assembly (IDS). A different surgical instrument 12 is removably mountable to each IDS. As best seen in FIG. 3, each instrument 12 includes an elongate shaft 106, which is preferably rigid but which may be flexible or partially flexible in alternative systems. An end effector 108 is positioned at the distal end of shaft 106, and a base assembly or adapter assembly 110 is at the proximal end.

Instrument and IDS configurations suitable for use with the disclosed inventions will next be described, but it should be understood that these are given by way of example only. The disclosed manipulator may be used with various configurations of instruments and instrument drive systems. More particularly, while the receiver/IDS described here is configured to drive pitch and jaw motion of an articulated surgical instrument, in alternative embodiments the receiver/IDS may have less functionality. In some alternative configurations, it may serve simply to receive an instrument and to drive jaw open/close operations. In other configurations, it may be configured, along with the instrument, to actuate a roll function of the instrument tip relative to the shaft of the instrument.

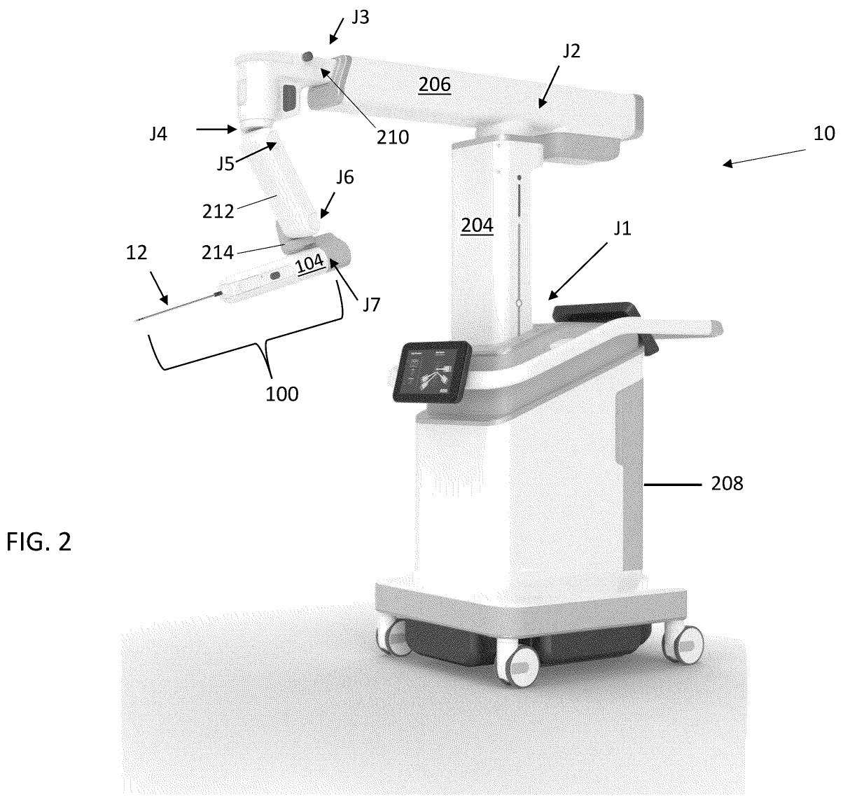

The instrument depicted in the drawings is the type described in Applicant's commonly-owned co-pending application published as US 2020/0375680, entitled Articulating Surgical Instrument, which is incorporated herein by reference. It makes use of four drive cables two of which terminate at one of the jaw members and the other two of which terminate at the other jaw member. This can be two cables looped at the end effector (so each of the two free ends of each cable loop is at the proximal end) or it can be four individual cables. As described in the co-pending application, the tension on the cables is varied in different combinations to effect pitch and yaw motion of the jaw members and jaw open-close functions. Other instruments useful with the system will have other numbers of cables, with the specific number dictated by the instrument functions, the degrees of freedom of the instrument and the specific configuration of the actuation components of the instrument. Note that in this description the terms “tendon,” “wire,” and “cable” are used broadly to encompass any type of tendon that can be used for the described purpose. The surgical instrument's drive cables extend from the end effector 108 through the shaft 106 (FIG. 2) and extend into the adapter assembly 110 where they are coupled to mechanical actuators. A more detailed description is given in Applicant's co-pending application published as US 2021/169595, which is incorporated herein by reference, but a general configuration of these actuators with respect to the adapter assembly will be provided here.

The adapter assembly 110 (which will also be referred to as the “adapter”) may include an enclosed or partially enclosed structure such as a housing or box, or it may be a frame or plate. The exemplary adapter 110 shown in the drawings includes mechanical input actuators 112 exposed to the exterior of the surgical instrument 102. In FIG. 3, two mechanical input actuators 112 are exposed at a first lateral face of the adapter 110. A second two mechanical input actuators 112 (not visible in FIG. 3) may be exposed at the second, opposite, lateral face of the adapter 110, preferably but optionally in a configuration identical or similar to the configuration shown in FIG. 3.

Each of the mechanical input actuators 112 is moveable relative to the adapter 110 between first and second positions. In the specific configuration shown in the drawings, the actuators are longitudinally moveable relative to the housing between a first (more distal) position and a second (more proximal) position such as that shown in FIG. 3. The direction of motion, however, is not required to be longitudinal and can extend in any direction.

In this configuration, the adapter thus has four drive inputs, one for each of the input actuators 112, exposed to its exterior. The illustrated adapter has two parallel planar faces, with two of these inputs positioned on each of the faces. While it may be preferred to include the inputs on opposite sides of the proximal body, other arrangements of inputs on multiple faces of the proximal body can instead be used. Each of these configurations advantageously arranges the drive inputs to maximize the distance between control inputs, minimizing stresses in the sterile drape that, in use, is positioned between the proximal body and the receiver 104. Co-pending US 2021/169595 includes further description of the adapter shown in FIG. 3.

The IDS 104 at the end of each manipulator 10 has an open position (shown in FIG. 3) in which it removably receives the adapter 110 of a corresponding instrument 12, to form an assembly 100. After the adapter 110 is placed within the IDS, the IDS is moved to the closed position shown in FIG. 2, capturing the adapter 110. In this position, the drive inputs 112 of the adapter can engage with corresponding drive outputs 114 of the IDS. As described in detail in co-pending US 2021/169595, user input at the input devices 16, 18 commanding jaw open-close, pitch or yaw articulation etc. of the instrument causes electromechanical actuators in the IDS to move the drive outputs 114. The motion of those drive outputs moves corresponding ones of the adapter's drive inputs 112, altering tension on the instrument's drive cables in a manner that causes the desired motion at the instrument's end effector.

### Robotic Manipulator

A first aspect of the disclosed inventions relates to fundamental components required for the kinematic structure of a redundant surgical manipulator to allow accurately replicated motion by maintaining high dexterity and sensitivity across the overall surgical workspace. This aspect is motivated by the need for a surgical manipulator that can be utilized for multiple surgical indications without degradation in performance due to its achievable workspace. The locations of the joints and their offsets in the kinematic chain of a surgical manipulator play a crucial role in their ability to replicate the surgeon's motion. In addition, the spatial configuration of the patient, the manipulator and the constraint as well as the dimension of the surgical workspace greatly change between procedures. This makes some surgical manipulators better suited only for certain types of indications. However, it is desirable in surgery to operate over a broad range of MIS procedures without degradation in workspace.

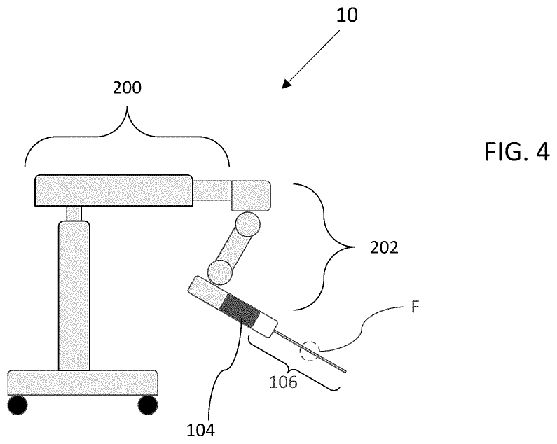

Turning to the schematic view of the robotic manipulator shown in FIG. 4, the manipulator kinematic structure includes three primary components. Each component plays a different role in the overall instrument motion which, during laparoscopic motion of the instrument 106, must occur while algorithmically constraining the motion at the fulcrum/RCM F, which corresponds to the incision site, the position of which is calculated as discussed above. The first two are integral components of the manipulator: a base structure 200, formed of the proximal joints J1, J2 and J3 (discussed below), and a distal structure 202, formed of the distal joints J4-J7 (discussed below). The base structure functions primarily to place the distal structure above the surgical site by means of translation motions, whereas the primary purpose of the distal structure is to produce additional three DOF changes in the orientation of the instrument tip. Thus, during operation of the manipulator, motion of the proximal joints of the base structure 200 primarily governs position of the distal structure in three degrees of freedom, whereas motion of the distal joints 202 primarily governs orientation of the instrument tip in 3+, and preferably 4+ degrees of freedom. These two parts are discussed in further detail below. The third part is the instrument structure which in preferred embodiments is the IDS 104 or an alternative component which operates to control the instrument's distal joint(s). This part may optionally also include joints to allow for off-axis instrument placement.

The joints of base structure 200 and distal structure 202 will be further discussed in connection with FIG. 2. The base 200 structure is configured to reposition the distal structure 202 in 3 DOFs using a sequence of prismatic-revolute-prismatic joints, also referred to herein as the proximal joints. These motor-driven proximal joints are identified in the drawings as J1, J2 and J3, with J1 being a prismatic joint, J2 being a revolute joint, and J3 being a prismatic joint. The prismatic joints may alternatively be referred to as rectilinear translation joints. As can be seen in the drawing, these joints are arranged in sequence, without intervening joints that would influence positioning of the distal structure 202.

The robotic manipulator includes a vertical column assembly 204 and a horizontal boom assembly 206. The vertical column assembly 204 preferably extends from a cart 208 having wheels that allow the robotic manipulator to be repositioned within the operating room by rolling it across the floor. Horizontal boom assembly 206 supports the distal structure 202 at its distal end.

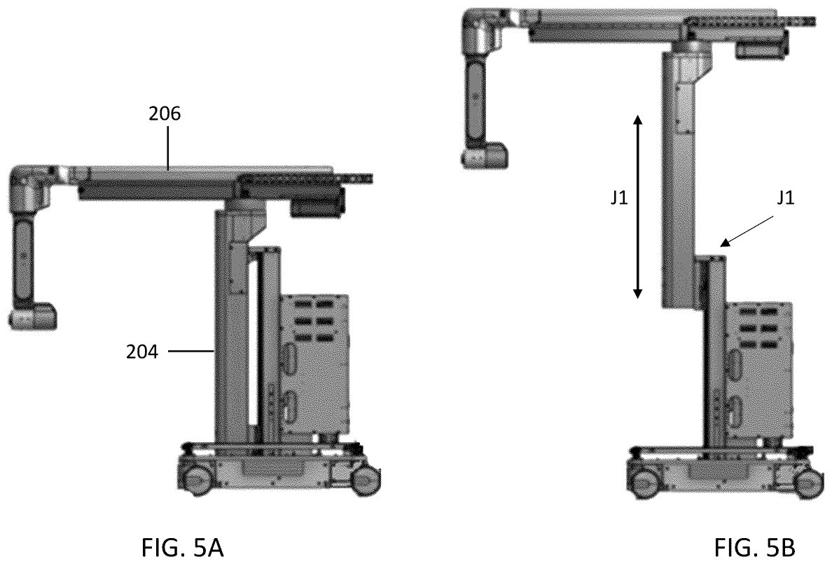

Joint J1 extends and retracts the vertical column assembly 204 relative to the cart 208 along a vertical axis as illustrated in FIGS. 5A and 5B. This motion functions to raise and lower the horizontal boom assembly 206 as shown. J1 may be configured to permit between 650-800 mm of extension of the vertical column assembly 204, and more preferably between 700-750 mm.

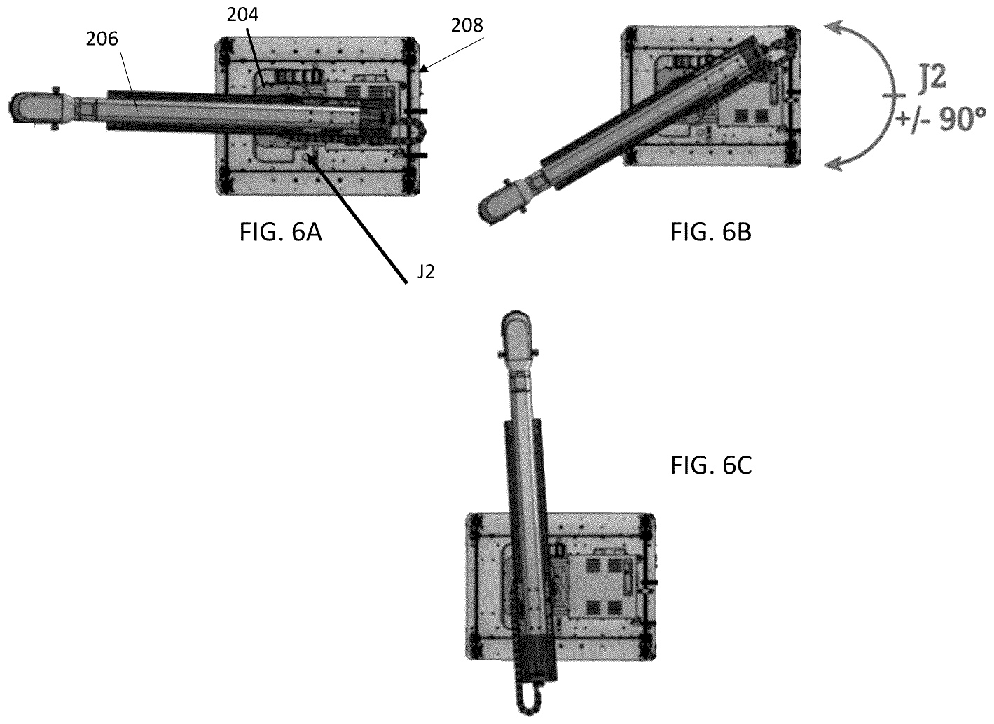

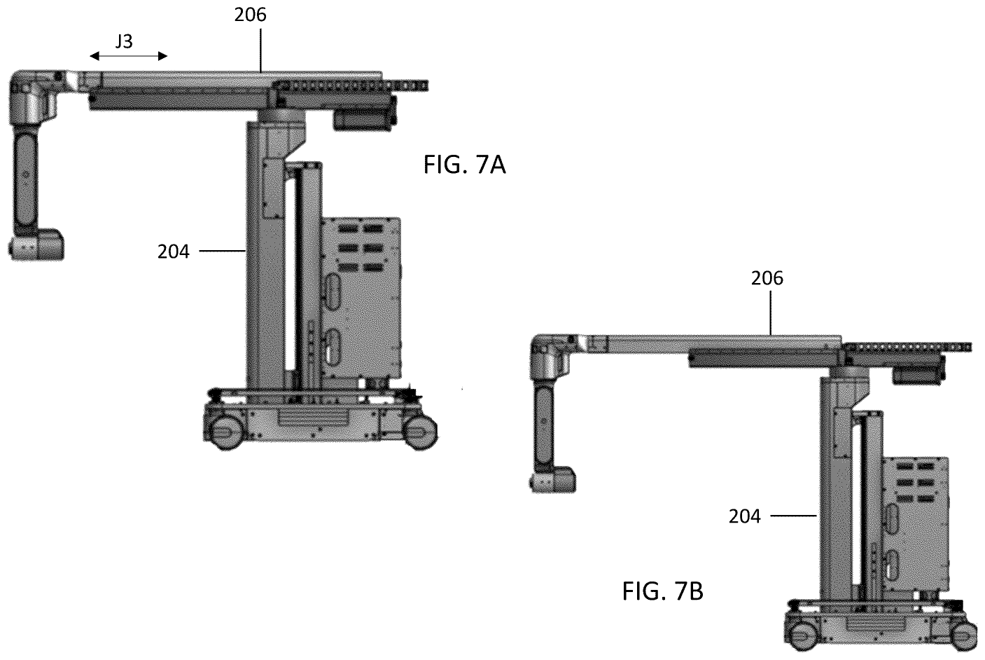

Joint J2 pivots the horizontal boom assembly 206 relative to a vertical axis of the column assembly 204 as illustrated in FIGS. 6A-6C. In preferred embodiments, J2 is configured to permit up to 180 degrees of rotation (90 degrees in each direction). Joint J3 extends and retracts a part 210 of horizontal boom assembly 206 along a horizontal axis. Sec FIGS. 7A-7B. J3 may be configured to permit between 650-800 mm of extension of the horizontal boom assembly 206, and more preferably between 700-750 mm.

The distal structure 202 is configured to produce changes in orientation of the instrument tip in an additional 3DOFs. The distal structure has at least four motor-driven joints, also referred to as the distal joints, arranged to generate redundancy in joint space. The locations of the joints allow for uniform dexterity and minimize effect of singularities and joint limits along the workspace. The architecture of distal structure 202 includes a first link 212 coupled to the distal end of the horizontal boom assembly 206, and a second 214 at the distal end of the first link 212. See FIG. 2. Second link 214 supports the IDS 104, as well as the force/torque sensor (not shown), which is situated just proximal to the IDS and distal to joint J7 (discussed below). As discussed above, the feedback from the force/torque sensor is used in the control of motion of the arm 10 to ensure that movement of the instrument is algorithmically constrained about the fulcrum at the incision site.

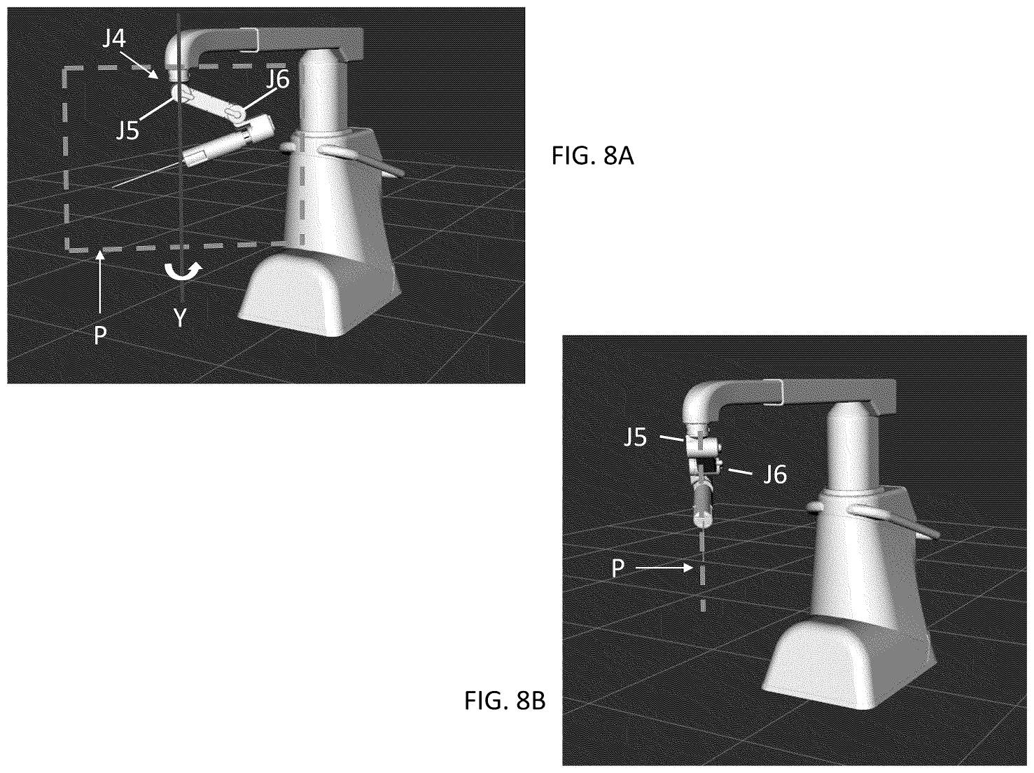

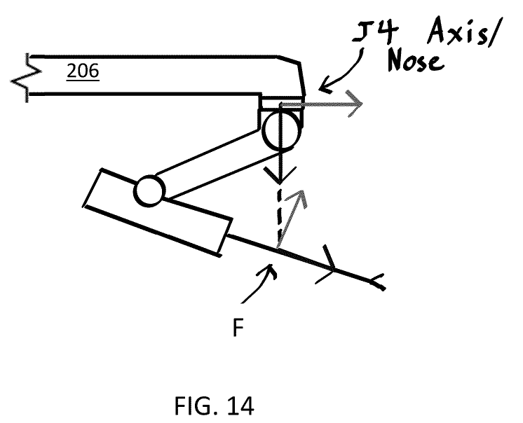

FIGS. 8A and 8B illustrate, using dashed lines, the plane P containing the longitudinal axis of the instrument shaft 106 (which is preferably a vertical plane). It is useful to describe the distal joints with respect to the plane P. The preferred kinematic configuration of the distal structure 202 includes four motor-driven distal joints identified as J4-J7. J4 is a revolute joint disposed between the distal end of the horizontal boom assembly 206 and link 212 (labeled in FIG. 2). Actuation of J4 produces rotation of the link 212, and thus distal structure 202, about a vertical axis Y, the axis of joint J, which lies within the plane P. Said another way, when joint J4 is driven, the plane P containing the longitudinal axis of the instrument shaft rotates about a vertical axis. In operation, J4 is preferably the primary contributor of yaw motion of the instrument shaft. In FIGS. 8A and 8B, it can be seen that the Y axis is the yaw axis about which rotation of the plane P containing the instrument shaft occurs. Yaw axis Y intersects the longitudinal axis of the instrument shaft and, preferably during operation of the arm, intersects the RCM defined at the fulcrum, as shown in FIG. 14, in order to optimize the dexterity of the system.

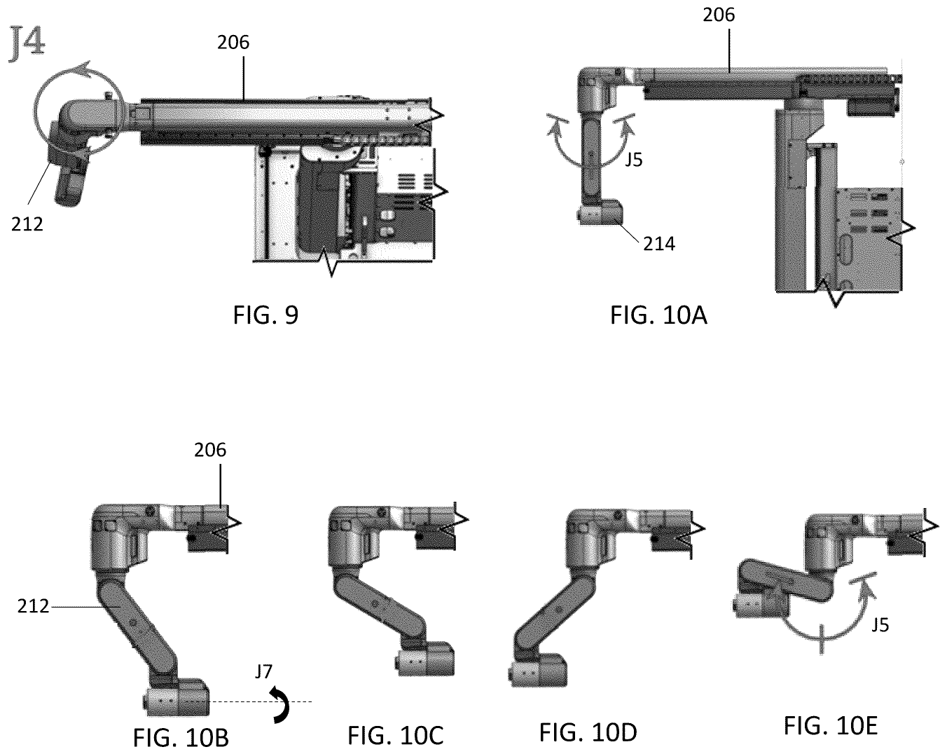

In a preferred embodiment, J4 is configured for unrestricted rotation as depicted in FIG. 9, although in alternative embodiments it may be configured for less than unrestricted rotation, such as 360 degree or 540 degree rotation.

Distally adjacent to J4 is a series of revolute joints, J5 and J6, which are coplanar and thus have parallel axes of rotation. These joints are located at opposite ends of link 212. The rotational axes of J5 and J6 have an orientation that is perpendicular to the plane P.

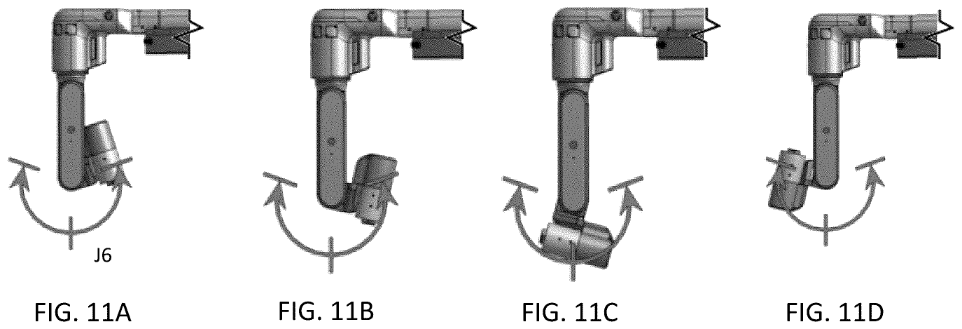

J5 and J6 operate to move the longitudinal axis of the instrument within plane P. Operation of these joints can be used to effectuate pitch motion of the instrument shaft. During pitch motion of the instrument shaft 106 through operation of J5 and/or J6, the longitudinal axis of the instrument shaft remains within plane P. FIGS. 10A-10E show the distal joints in a series of position resulting from actuation of joint J5, and FIGS. 11A-11D show the distal joints in a series of positions resulting from actuation of joint J6. As shown, when J5 is actuated, it causes planar pitch motion of link 212 about the J5 axis, and when J6 is actuated it pivots the distal link 214 about the J6 axis, which is parallel to the J5 axis. Operation of J5 and J6 can thus be used to generate pitch motion of the distal link 214 (and consequently the instrument mounted to the link 212 via the IDS). Operational ranges for J5 and J6 are +/−110 degrees, and, more preferably, +/−90 degrees. It should be noted with regard to the joint ranges given in this application that operational ranges used in practice may be less than the joints' functional limits. In some cases, the control algorithms may artificially reduce the range of motion of all or certain joints. This may be done, for example, to accommodate smooth motion of other joints. Such operational limits may be configuration dependent, varying based on where the instrument or the relevant joint is in the workspace.

The final joint in the series of distal joints is revolute joint J7. Operation of J7 produces roll motion of the instrument shaft about an axis that is coincident with the longitudinal axis of the instrument shaft. The rotational axis of J7 has an orientation that is perpendicular to the orientations of pivot axes of each of J5 and J6 as can be seen in FIG. 10B, but it does not intersect with them. J7 may be configured for unrestricted rotation or for less than unrestricted rotation, such as 360 degree or 540 degree rotation. When J7 is operated, the force torque sensor that is just proximal to the IDS, as well as the IDS and the instrument, are caused to rotate.

In some cases, the arm may be provided without J7. This might be suitable in cases, for example, where the surgical instrument is one that has an end effector configured to axially roll relative to the instrument shaft using input from the IDS or actuators carried by the instrument adapter.

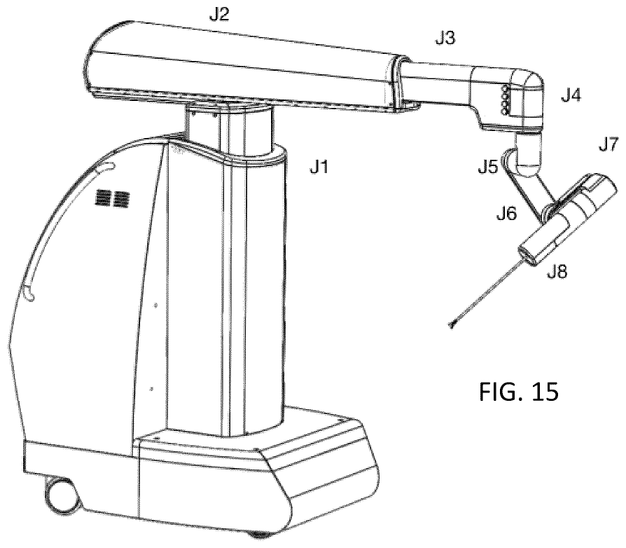

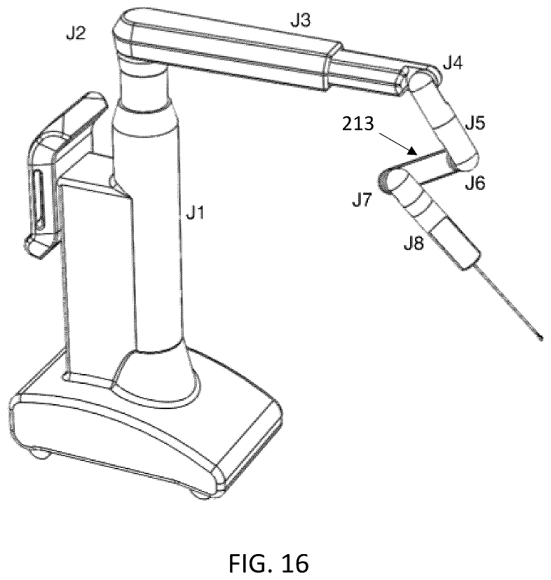

While the disclosed arm includes 7 DOFs, alternative configurations may include more than 7 DOFs. See for example, FIGS. 15, in which a prismatic joint is added before J7, allowing axial translation of the instrument, IDS and the most distal joint J8, which has the characteristics of the J7 joint described for the first embodiment. This translational joint can be particularly useful for instrument exchange, allowing withdrawal of the instrument along its longitudinal axis without the need to change the other joint positions. Another example is shown in FIG. 16, which shows an additional link 213 between links 212 and 213, with an additional rotational joint, here J7, at its distal end. In this embodiment, there is no rotational joint about a vertical axis other than J2. In other words, the J4 joint of the original embodiment is eliminated. However, this embodiment may be made a 9 DOF arm by adding such an embodiment at the distal end of the horizontal boom member, similar to joint J4 of the first embodiment.

### Control

A second aspect of the disclosed inventions relates to control methods that apply to the above-described kinematic framework for enhancing dexterity and sensitivity across the manipulator workspace independent of the preoperative configuration of the arm and the patient. This aspect is motivated by the need for more consistent results within the same procedure and between surgeries. For many prior art surgical manipulators, the ability of the manipulator to replicate the surgeon's input motion is highly dependent on the manipulator's workspace configuration. However, surgeons are better able to evaluate and improve their own performance if the accuracy of motion replication does not change within the arm workspace. This means that the arm itself should not affect the performance of the surgeon but instead it should be “transparent” and replicate the motion as exactly as possible. An arm with enhanced dexterity across the full workspace will allow the surgeon to have more repeatable and predictable results.

With currently available systems, issues related to variable dexterity across the workspace are compensated for by extensive training. During training, surgeons learn the optimal preoperative placement of the arm and patient for each procedure and how to visually compensate for motion inaccuracies. However, this requires many hours of training for the surgeon to master the device and causes variability of results between surgeons. In addition, as autonomous or semi-autonomous tasks become more ubiquitous within the surgical field, dexterity and sensitivity of the manipulator arm become increasingly fundamental for the arm to independently understand and act in the surgical environment. The configuration of the arm 10 provides increased dexterity across a broader workspace than prior algorithmically-controlled systems, making it suitable for a broad range of surgical indications.

As discussed above, because the arm 10 is designed without architecture that mechanically constrains its motion relative to an RCM, motion of the arm in response to user input is algorithmically controlled relative to the RCM location at the incision site, that is determined using the force-torque sensor or other methods. Control may thus rely on feedback from the force/torque sensor disposed near J7 to determine the location of the incision along instrument shaft 106, or by other RCM-determining methods including, without limitation, those discussed in the Background. In preferred operation of the arm, ensuring that shaft motion occurs about the RCM is the highest priority of the robotic controller, with a second priority being moving the instrument tip to the target position (determined based on the input from the user).

In typical use of the arm, all of the joints J1-J7 are active for use, when needed, to move the instrument laparoscopically relative to the incision site (i.e. to move the instrument shaft in pitch, yaw, roll, and along its insertion axis, which coincides with the longitudinal axis of the shaft) in response to user input instructing the system as to the desired instrument position and orientation. In controlling operation of the arm to achieve that position and orientation, the system preferably prioritizes operation of the distal joints J4-J7 over that of the proximal joints J1-J3. To allow the manipulator to adjust for multiple task requirements all joints are considered active during preoperative placement and operation.

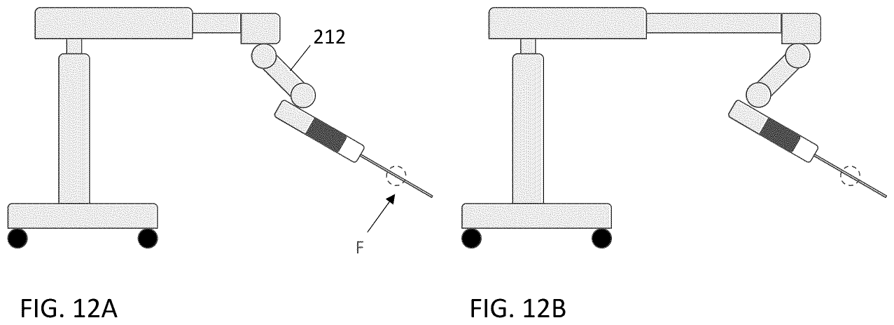

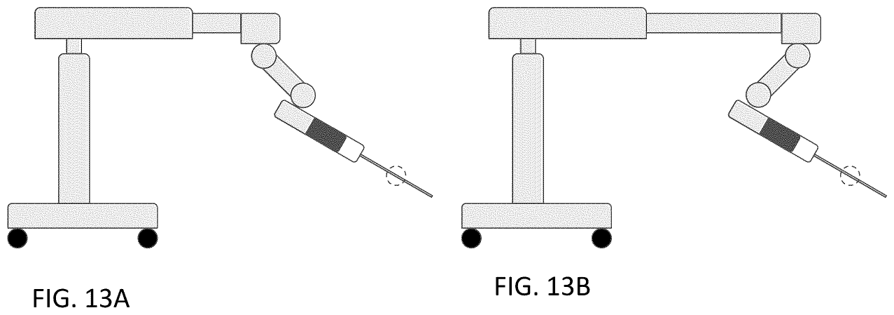

One advantage of arm 10 over certain prior art arms using algorithmically controlled RCM is that not only can the approach angle (i.e. the angle between the shaft of the instrument and the horizontal floor) be used to control the system, but the angle of link 212 can also be used due to the redundancy J5 and J6 provide. Consequently, configuration variables for moving the instrument's distal end to the desired position and orientation include roll position, redundant angle, approach angle, and yaw position. The relative distance between the distal joints and the constraint at the trocar may be adjusted using redundancy without producing motion of the instrument shaft. FIGS. 12A and 12B illustrate two configurations of joint positions, each of which results in the same approach angle to the patient through different orientations of link 212. As will be understood from comparing the two figures, the position of link 212 on the left results in less extension at joint J3, while maintaining the same approach angle and RCM point. In alternative contexts, the link angle 212 can be adjusted while maintaining the same approach angle and preserving the RCM point in a way that reduces the joint extension at joint J. FIGS. 13A and 13B additionally show that the instrument can be translated along its longitudinal axis while crossing the singularity that occurs when J5 and J6 are vertically aligned with one another.

The redundant angle can be configured based on performance metrics, such as minimization of joint limits, collision avoidance, avoidance of singularities, etc. For example, the FIG. 12B and FIG. 13A arrangements may result from prioritization of joint limit avoidance tasks for joints J1 and J3, respectively. FIG. 14 illustrates one way in which the control algorithms may also prioritize minimal lateral displacement of J3. In this example, J3 lateral displacement may be limited to prevent a point at or near the distal part of the horizontal boom assembly from moving beyond a predetermined point, such as the fulcrum/RCM F. As one example, the point at/near the distal part of the horizontal boom may be the axis of joint J4.

An additional benefit of arm 10 is increased responsiveness to hand guiding operations. During a hand-guiding operation, a user's hand applies force to the arm 10 to urge the arm towards the desired position. Feedback in the force/torque sensor disposed near J7 is used by the system to calculate the direction of movement desired by the user, and the system activates motors in the joints to aid the user in repositioning the arm. In alternative arms which do not use such a force/torque sensor, this may be calculated using force/torque sensors positioned at joints of the arm, and/or using motor currents at the joints. Because the arm 10 uses redundant joints at J5-J6, less motion is required from J1-J3 during hand-guiding, and the system thus demonstrates increased sensitivity to hand-guiding input.

While certain embodiments have been described above, it should be understood that these embodiments are presented by way of example, and not limitation. It will be apparent to persons skilled in the relevant art that various changes in form and detail may be made therein without departing from the scope of the invention characterized by the claims. This is especially true in light of technology and terms within the relevant art(s) that may be later developed. Moreover, features of the various disclosed embodiments may be combined in various ways to produce various additional embodiments.

All patents, patent applications and printed publications referred to above, including for purposes of priority, are incorporated herein by reference.

We claim:

1. A surgical robotic manipulator comprising:
a proximal assembly comprising a vertical column and a horizontal boom, the proximal assembly including:
a first prismatic joint operable to extend and retract the vertical column to move the boom along a first vertical axis between first and second positions,
a proximal revolute joint between the vertical column and the horizontal boom, the proximal revolute joint operable to rotate the horizontal boom relative to the vertical column about the first vertical axis,
a second prismatic joint operable to extend and retract the horizonal boom along a horizontal axis; and
a distal assembly disposed at a distal end of the horizontal boom, the distal assembly comprising:
a first link,
a first distal revolute joint between the horizontal boom and the first link;
a pair of second distal revolute joints, a first one of said pair of distal revolute joints disposed at a proximal end of the first link and having a first rotational axis, and a second one of said pair of distal revolute joints disposed at a distal end of the first link and having a second rotational axis, the first rotational axis and the second rotational axes being coplanar axes;
a second link coupled to the second one of the pair of distal revolute joints; and
an instrument receiver on the second link, the instrument receiver configured to receive and support a surgical instrument such that a longitudinal axis of the surgical instrument lies within a vertical plane;
wherein the first distal revolute joint is operable to rotate the first link about a second vertical axis, causing rotation of the vertical plane containing the second vertical axis; and
wherein the second distal revolute joints are operable to rotate the instrument receiver such that the longitudinal axis moves within the vertical plane.

2. The surgical robotic manipulator of claim 1, further including a third distal revolute joint distal to the second distal revolute joints, the third distal revolute joint operable to rotate the instrument receiver about the longitudinal axis.

3. The surgical manipulator of claim 2, wherein the third distal revolute joint is on the second link.

4. The surgical manipulator of claim 1, wherein the instrument receiver is an instrument drive system configured to receive and actuate a surgical instrument having a distal tip articulatable in at least two degrees of freedom.

5. The surgical robotic manipulator of claim 4, further including a third distal revolute joint distal to the second distal revolute joints, the third distal revolute joint operable to rotate the instrument drive system relative to the longitudinal axis.

6. The surgical robotic manipulator of claim 1, wherein the second distal revolute joints are operable to rotate the instrument receiver to cause pitch motion of the longitudinal axis.

7. The surgical robotic manipulator of claim 1, wherein the first distal revolute joint is operable to rotate the instrument receiver to cause yaw motion of the longitudinal axis.

8. The surgical robotic manipulator of claim 1, wherein the manipulator is configured such that operation of the first one of said pair of distal revolute joints rotates the first link about the first rotational axis

9. The surgical robotic manipulator of claim 8, wherein the manipulator is configured such that operation of the second one of the pair of distal revolute joints rotates the second link relative to the second rotational axis.

10. The surgical manipulator of claim 1, wherein the first link has a first position in which the manipulator positions the surgical instrument with a first approach angle, and a second position in which the manipulator positions the surgical instrument with the first approach angle, wherein the first link is moveable between the first position and the second position without moving the surgical instrument out of the first approach angle.
